(19) United States
(12) Patent Application Publication
(10) Pub. No.: US 2024/0052390 A1
(43) Pub. Date: Feb. 15, 2024

(54) FERMENTED SUPERNATANT OF BANGIA FUSCO-PURPURE AND LACTOBACILLUS WITH ALPHA-GLUCOSIDASE INHIBITORY ACTIVITY AND USE THEREOF

(71) Applicant: JIMEI UNIVERSITY, Xiamen (CN)

(72) Inventors: Zedong JIANG, Xiamen (CN); Yuting DONG, Xiamen (CN); Hui NI, Xiamen (CN); Xiping DU, Xiamen (CN); Yanhong CHEN, Xiamen (CN); Yuanfan YANG, Xiamen (CN); Zhipeng LI, Xiamen (CN)

(73) Assignee: JIMEI UNIVERSITY, Xiamen (CN)

(21) Appl. No.: 18/282,238

(22) PCT Filed: Mar. 16, 2022
(86) PCT No.: PCT/CN2022/081079

§ 371 Date: Sep. 15, 2023

(30) Foreign Application Priority Data
Mar. 16, 2021 (CN) 202110281294.X

(51) Int. Cl. C12P 19/04; A61K 31/715

(52) U.S. Cl. CPC C12P 19/04; A61K 31/715

(57) ABSTRACT

A fermented supernatant of Bangia fusco-purpure and Lactobacillus with an alpha-glucosidase inhibitory activity is provided, where a preparation method for the fermented supernatant of Bangia fusco-purpure and Lactobacillus includes the following steps of: S1: mixing Bangia fusco-purpure with water in a mass/volume ratio of 4.45%-5% to obtain a Bangia fusco-purpure suspension; S2: adding glucose in a mass/volume ratio of 2% to the Bangia fusco-purpure suspension, followed by pasteurization; S3: inoculating Lactobacillus delbrueckii or Lactobacillus plantarum in a mass/volume ratio of 4% to obtain a mixed solution of Bangia fusco-purpure and Lactobacillus; S4: fermenting the mixed solution of Bangia fusco-purpure and Lactobacillus at 37° C. for 48 hours while standing to obtain a mixed fermentation broth of Bangia fusco-purpure and Lactobacillus; and S5: removing algal residues and Lactobacillus thalli via centrifuging the mixed fermentation broth of Bangia fusco-purpure and Lactobacillus, to obtain the fermented supernatant of Bangia fusco-purpure and Lactobacillus.

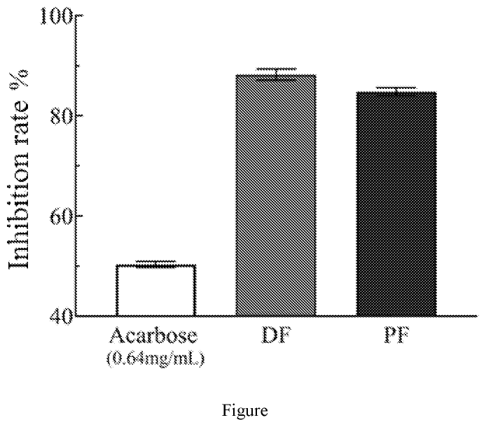

### CROSS REFERENCE TO THE RELATED APPLICATIONS

This application is the national phase entry of International Application No. PCT/CN2022/081079, filed on Mar. 16, 2022, which is based upon and claims priority to Chinese Patent Application No. 202110281294.X, filed on Mar. 16, 2021, the entire contents of which are incorporated herein by reference.

### TECHNICAL FIELD

The present disclosure belongs to the technical field of biological resource development, and in particular relates to a fermented supernatant of Bangia fusco-purpure and Lactobacillus and use thereof.

### BACKGROUND

Diabetes mellitus is the third largest chronic non-infectious disease following cardiovascular diseases and tumors. Wherein more than 90% of diabetic patients have type 2 diabetes mellitus (T2DM). An alpha-glucosidase inhibitor can competitively inhibit the activity of alpha-glucosidase in the small intestine, and delay or inhibit the absorption of glucose in the intestinal tract, thereby effectively lowering the peak of postprandial blood glucose and regulating the level of blood glucose in the body. The alpha-glucosidase inhibitor plays a crucial role in a process of inhibiting postprandial hyperglycemia, and has always been an ideal drug for the treatment of type 2 diabetes mellitus. Acarbose is a glycosidase inhibitor widely used in clinical practice at present, and has a good effect on the control of blood glucose, but long-term administration has some side effects such as liver damage and intestinal obstruction. Therefore, search for a natural, safe and economical alpha-glucosidase inhibitor is a better way to delay or assist the treatment of type 2 diabetes mellitus. Currently, research on alpha-glucosidase inhibitors mainly focuses on chemical synthesis or extraction of alpha-glucosidase inhibitors from natural substances, but the alpha-glucosidase inhibitors obtained by these methods all have certain side effects, such as causing diseases of the liver, intestine, stomach and the like; and meantime, these inhibitors also have the problems such as low extraction amount and high cost. However, a method of food lactic acid bacterium fermentation is used to produce a product with an alpha-glucosidase inhibitory activity, which has low cost and high safety.

Bangia fusco-purpure, also known as Hongmaocai, Hongmianzao, etc., is a unique economic red algae resource in Fujian coastal areas of China, and fresh and delicious Bangia fusco-purpure has remarkable effects of lowering blood pressure, improving blood deficiency, nourishing yin and falling fire, and preventing vascular diseases. A supernatant extracted from Bangia fusco-purpure and a fermented supernatant of Bangia fusco-purpure and Lactobacillus without optimization of a fermentation process used in the prior art have been shown with low inhibitory effects (lower than 40%) on the activity of alpha-glucosidase. Therefore, it cannot yet replace alpha-glucosidase inhibitors that are chemically synthesized or extracted from natural substances.

### SUMMARY

In view of this, in one aspect, the present disclosure provides a fermented supernatant of Bangia fusco-purpure and Lactobacillus that has an alpha-glucosidase inhibitory activity, which has the advantages of low cost, high safety, and high inhibitory activity, and can replace alpha-glucosidase inhibitors that are chemically synthesized or extracted from natural substances in the prior art.

The present disclosure achieves the above first inventive object by the following technical solution:
provided is a fermented supernatant of Bangia fusco-purpure and Lactobacillus with an alpha-glucosidase inhibitory activity, wherein a preparation method for the fermented supernatant of Bangia fusco-purpure and Lactobacillus includes the following steps of:

S1: mixing Bangia fusco-purpure with water in a mass/volume ratio of 4.45%-5% to obtain a Bangia fusco-purpure suspension;

S2: adding glucose in a mass/volume ratio of 2% to the Bangia fusco-purpure suspension, followed by pasteurization;

S3: inoculating Lactobacillus delbrueckii or Lactobacillus plantarum in a mass/volume ratio of 4% to obtain a mixed solution of Bangia fusco-purpure and Lactobacillus;

S4: fermenting the mixed solution of Bangia fusco-purpure and Lactobacillus at 37° C. for 48 hours while standing to obtain a mixed fermentation broth of Bangia fusco-purpure and Lactobacillus; and

S5: removing algal residues and Lactobacillus thalli via centrifuging the mixed fermentation broth of Bangia fusco-purpure and Lactobacillus, to obtain the fermented supernatant of Bangia fusco-purpure and Lactobacillus.

Further, the preparation method for the fermented supernatant of Bangia fusco-purpure and Lactobacillus further includes the step of:

S0: grinding Bangia fusco-purpure with liquid nitrogen before mixing Bangia fusco-purpure with water.

Further, the step S1 includes mixing Bangia fusco-purpure with water in a mass/volume ratio of 5% to obtain a Bangia fusco-purpure suspension, and the step S3 includes inoculating Lactobacillus delbrueckii in a mass/volume ratio of 4% to obtain a mixed solution of Bangia fusco-purpure and Lactobacillus, wherein the fermented supernatant of Bangia fusco-purpure and Lactobacillus is a fermented supernatant of Bangia fusco-purpure and Lactobacillus delbrueckii, having an inhibition rate of 88.2% toward alpha-glucosidase.

Further, the step S1 includes mixing Bangia fusco-purpure with water in a mass/volume ratio of 4.45% to obtain a Bangia fusco-purpure suspension, and the step S3 includes inoculating Lactobacillus plantarum in a mass/volume ratio of 4% to obtain a mixed solution of Bangia fusco-purpure and Lactobacillus, wherein the fermented supernatant of Bangia fusco-purpure and Lactobacillus is a fermented supernatant of Bangia fusco-purpure and Lactobacillus plantarum, having an inhibition rate of 84.8% toward alpha-glucosidase.

A second inventive object of the present disclosure is to provide use of a fermented supernatant of Bangia fusco-purpure and Lactobacillus that has an alpha-glucosidase inhibitory activity, and the technical solution thereof is as follows:
use of the fermented supernatant of Bangia fusco-purpure and Lactobacillus achieving the first inventive object in the preparation of an alpha-glucosidase inhibitor.

The fermented supernatant of Bangia fusco-purpure and Lactobacillus provided by the present disclosure is a natural, safe and economical alpha-glucosidase inhibitor, has an alpha-glucosidase inhibition rate greater than 80%, and can replace alpha-glucosidase inhibitors that are chemically synthesized or extracted from natural substances in the prior art.

### BRIEF DESCRIPTION OF THE DRAWING

The figure is a comparison diagram of the inhibition test results of alpha-glucosidase by fermented supernatants obtained in Example 1 and Example 2 of the present disclosure and a positive control acarbose.

In the figure, Acarbose is acarbose, DF represents a fermented supernatant of Bangia fusco-purpure and Lactobacillus delbrueckii, and PF represents a fermented supernatant of Bangia fusco-purpure and Lactobacillus plantarum.

### DETAILED DESCRIPTION OF THE EMBODIMETNS

### EXAMPLE 1: PREPARATION OF A FERMENTED SUPERNATANT OF BANGIA FUSCO-PURPURE AND Lactobacillus delbrueckii, AND ALPHA-GLUCOSIDASE INHIBITION RATE TEST

### Preparation:

Bangia fusco-purpure was ground with liquid nitrogen;

50 g of the ground Bangia fusco-purpure was taken to be mixed with 1000 ml of water to obtain a Bangia fusco-purpure suspension;

20 g of glucose was added to the Bangia fusco-purpure suspension in a mass/volume ratio, followed by pasteurization;

40 g of Lactobacillus delbrueckii was inoculated in a mass/volume ratio to obtain a mixed solution of Bangia fusco-purpure and Lactobacillus delbrueckii;

the mixed solution of Bangia fusco-purpure and Lactobacillus delbrueckii was fermented at 37° C. for 48 hours while standing to obtain a mixed fermentation broth of Bangia fusco-purpure and Lactobacillus delbrueckii;

the mixed fermentation broth of Bangia fusco-purpure and Lactobacillus delbrueckii was centrifuged to remove algal residues and Lactobacillus thalli to obtain the fermented supernatant of Bangia fusco-purpure and Lactobacillus delbrueckii.

Test:

100 μl of the fermented supernatant of Bangia fusco-purpure and Lactobacillus delbrueckii and 250 μl of an alpha-glucosidase solution were taken to be mixed and incubated in a water bath at 37° C. for 10 min, and 250 μl of a substrate solution was added thereto to start a reaction, and 250 μl of a 1 mol/L Na₂CO₃ solution was added to stop the reaction after the reaction was carried out at 37° C. for 15 min, and its absorbance value was measured at a wavelength of 405 nm. Acarbose was used as a positive control, the same volume of PBS buffer instead of the fermentation broth was used as a blank group, and the same volume of PBS buffer instead of the alpha-glucosidase solution was used as a background control group, and 3 parallel experiments were set for each experimental system.

A formula for calculating an inhibition rate was:

$$Inhibition rate ( \% ) = \frac{{A}_{1} - ( {A}_{2} - {A}_{3} )}{{A}_{1}} \times 100.$$

In the formula, A1: an absorbance after a reaction in which the PBS buffer was used instead of the fermented supernatant of Bangia fusco-purpure and Lactobacillus delbrueckii in the blank group; A2: an absorbance after a reaction in which a sample of the fermented supernatant of Bangia fusco-purpure and Lactobacillus delbrueckii or an acarbose aqueous solution was added in a sample group; and A3: an absorbance after a reaction in which the PBS buffer was used instead of the alpha-glucosidase solution in the background control group.

The positive control acarbose aqueous solution in this example has an IC50 of 0.64 mg/mL, and the purpose of setting the positive control was to test and compare the inhibitory effect of the fermented supernatant of Bangia fusco-purpure and Lactobacillus delbrueckii in this example with that of the positive control, and to provide a reference for others to how to ensure the consistency of the test when repeating this example.

As shown in the figure, an inhibition rate of the fermented supernatant of Bangia fusco-purpure and Lactobacillus delbrueckii in this example to alpha-glucosidase was 88.2%

### EXAMPLE 2: PREPARATION OF A FERMENTED SUPERNATANT OF BANGIA FUSCO-PURPURE AND Lactobacillus plantarum, AND ALPHA-GLUCOSIDASE INHIBITION RATE TEST

### Preparation:

Bangia fusco-purpure was ground with liquid nitrogen;

44.5 g of the ground Bangia fusco-purpure was taken to be mixed with 1000 ml of water to obtain a Bangia fusco-purpure suspension;

20 g of glucose was added to the Bangia fusco-purpure suspension in a mass/volume ratio, followed by pasteurization;

40 g of Lactobacillus plantarum was inoculated in a mass/volume ratio to obtain a mixed solution of Bangia fusco-purpure and Lactobacillus plantarum;

the mixed solution of Bangia fusco-purpure and Lactobacillus plantarum was fermented at 37° C. for 48 hours while standing to obtain a mixed fermentation broth of Bangia fusco-purpure and Lactobacillus plantarum;

the mixed fermentation broth of Bangia fusco-purpure and Lactobacillus plantarum was centrifuged to remove algal residues and Lactobacillus thalli to obtain the fermented supernatant of Bangia fusco-purpure and Lactobacillus plantarum.

Test:

A test method in this example was the same as that in Example 1. An inhibition rate of the fermented supernatant of Bangia fusco-purpure and Lactobacillus plantarum obtained in this example to alpha-glucosidase was 84.8%.

What is claimed is:

1. A fermented supernatant of Bangia fusco-purpure and Lactobacillus with an alpha-glucosidase inhibitory activity, wherein a preparation method for the fermented supernatant of Bangia fusco-purpure and Lactobacillus comprises the following steps of:
S1: mixing the Bangia fusco-purpure with water in a mass/volume ratio of 4.45%-5% to obtain a Bangia fusco-purpure suspension;
S2: adding glucose in a mass/volume ratio of 2% to the Bangia fusco-purpure suspension, followed by a pasteurization to obtain a pasteurized mixture;
S3: inoculating Lactobacillus delbrueckii or Lactobacillus plantarum in a mass/volume ratio of 4% to the pasteurized mixture to obtain a mixed solution of Bangia fusco-purpure and Lactobacillus;
S4: fermenting the mixed solution of Bangia fusco-purpure and Lactobacillus at 37° C. for 48 hours while standing to obtain a mixed fermentation broth of Bangia fusco-purpure and Lactobacillus; and
S5: removing algal residues and Lactobacillus thalli via centrifuging the mixed fermentation broth of Bangia fusco-purpure and Lactobacillus to obtain the fermented supernatant of Bangia fusco-purpure and Lactobacillus.

2. The fermented supernatant of Bangia fusco-purpure and Lactobacillus according to claim 1, wherein the preparation method for the fermented supernatant of Bangia fusco-purpure and Lactobacillus further comprises a step of:
S0: grinding the Bangia fusco-purpure with liquid nitrogen before mixing the Bangia fusco-purpure with the water.

3. The fermented supernatant of Bangia fusco-purpure and Lactobacillus according to claim 2, wherein in step S1, mixing the Bangia fusco-purpure with the water in the mass/volume ratio of 5% to obtain the Bangia fusco-purpure suspension, and in step S3, inoculating the Lactobacillus delbrueckii in the mass/volume ratio of 4% to obtain the mixed solution of Bangia fusco-purpure and Lactobacillus, the fermented supernatant of Bangia fusco-purpure and Lactobacillus is a fermented supernatant of Bangia fusco-purpure and Lactobacillus delbrueckii, and an inhibition rate of the fermented supernatant of Bangia fusco-purpure and Lactobacillus delbrueckii to alpha-glucosidase is greater than 80%.

4. The fermented supernatant of Bangia fusco-purpure and Lactobacillus according to claim 2, wherein in step S1, mixing the Bangia fusco-purpure with the water in the mass/volume ratio of 4.45% to obtain the Bangia fusco-purpure suspension, and in step S3, inoculating the Lactobacillus plantarum in the mass/volume ratio of 4% to obtain the mixed solution of Bangia fusco-purpure and Lactobacillus, the fermented supernatant of Bangia fusco-purpure and Lactobacillus is a fermented supernatant of Bangia fusco-purpure and Lactobacillus plantarum, and an inhibition rate of the fermented supernatant of Bangia fusco-purpure and Lactobacillus plantarum to alpha-glucosidase is greater than 80%.

5. A method of a use of the fermented supernatant of Bangia fusco-purpure and Lactobacillus according to claim 1 in a preparation of an alpha-glucosidase inhibitor.
